(19) United States
(12) Patent Application Publication
(10) Pub. No.: US 2015/0093449 A1
(43) Pub. Date: Apr. 2, 2015

(54) Method of Using Silicon Substituted Phosphates to Improve Healing of Bone and Soft Tissue

(71) Applicant: Donald H. Brancato, Saint Louis, MO (US)

(72) Inventor: Donald H. Brancato, Saint Louis, MO (US)

(21) Appl. No.: 14/504,798

(22) Filed: Oct. 2, 2014

Related U.S. Application Data
(63) Continuation-in-part of application No. 12/490,579, filed on Jun. 24, 2009, now abandoned.

(51) Int. Cl. A61K 33/42; A61L 31/02

(52) U.S. Cl. CPC A61K 33/42; A61L 31/028
USPC 424/601

(57) ABSTRACT

A method of use of silicon substituted phosphates that improves bone and soft tissue growth resulting in better adhesion and fixation of implanted prosthetics, which may be used as part of a tissue engineering scaffold for ligament replacement and results in improved healing of healing of damaged bone and soft tissue ulcers. The silicon substituted phosphates of various chemical composition and granular sizes may be used in direct application or in combination with other materials, not previously claimed in prior art used to treat the various medical conditions described.

### CROSS REFERENCE TO RELATED APPLICATIONS

This application is a continuation of U.S. patent application Ser. No. 12/490,579, filed Jun. 26, 2009, which is incorporated herein by reference in its entirety.

### TECHNICAL FIELD

The present invention relates to the use of inorganic chemicals classed as silicon substituted phosphates (SSPs), and more specifically to a method for using silicon substituted phosphate formulations to improve bone and soft tissue growth, resulting in better adhesion and fixation of implanted prosthetics, which may be used as part of a tissue engineering scaffold for ligament replacement, and results in improved healing of damaged bone and soft tissue ulcers.

### BACKGROUND OF THE INVENTION

When implanting prostheses such as total hip replacements, total knee replacements, and total shoulder replacements, surgeons commonly use bone cement, such as methyl-methacrylate, to fix the prosthetic bone inserts into the bone. Complications associated with implantation of prostheses include loosening and dislocation of the prosthetic which can result in pain and/or bone breakage and require revision surgery. One cause of loosening is inadequate prosthetic fixation to host tissue. Another cause of loosening is related to wear at the contact surface and the prosthetic which releases particulates creating a granulation layer at the interface of the host tissue and contact surface. Additionally, the granulation layer imposes an inability to fully transfer mechanical loads from bone to prostheses implanted with methyl-methacrylate bone cement. The mechanical failure of the granulation layer to fully transmit load from bone to prosthesis and the inflammation from particulates increase the risk potential for loosening. Another cause of loosening that has been observed when using bone cement is inadequate soft tissue to bone attachment as well as to metal resulting in particulates migrating in the granulation layer causing prosthetic loosening.

Another situation in which soft tissue to bone attachment is important is when tendons and/or ligaments must be replaced or reattached to bone after injury. In these cases, a scaffold is used to afford a mechanical structure and protection during the time dependent chemical delivery of the multiple factors necessary for cellular growth and differentiation, ultimately leading to the matured replacement structure being capable of functioning as engineered.

A situation in which soft tissue generation is important is in the case of ulcerous wounds. Health conditions and/or injury can cause decreased circulation in the extremities and even a small wound can result in an ulcer that is very difficult to heal.

Many formulations of silicon substituted phosphates (SSP) have been developed to eliminate bone cement for use in dental applications (see, e.g., Hench, U.S. Pat. No. 5,981,412). As a result, various formulations of SSP are commercially available and have been approved for use by the U.S. Food and Drug Administration. Dental implants have used SSP formulations in fixing dental prostheses that are implanted in the contaminated, high load alveolar ridge. SSP application is known to fix the prosthesis by promoting bone in-growth into the porous coated prosthesis and directing bonding bone to the prosthesis. In addition to the high bioactivity, the SSP formulations have shown no tendency to form fibrous tissue which would cause loosening.

The use of SSP implants in orthopedics has been limited because the SSP formulations that are known for promoting bone growth are mechanically weak with low fracture resistance. The bending strength is in the range which is insufficient for load bearing application. Thus the orthopedic use of SSP implants has been limited to non-load bearing applications or for buried implants loaded slightly or compressively.

Thus, what is sought is a method for using an SSP formulation for promoting bone and tissue growth in orthopedic applications which involve load bearing bones or ulcerous skin conditions which overcomes one or more of the shortcomings and limitations, set forth above.

### SUMMARY OF THE INVENTION

What is disclosed is a method for using a silicon substituted phosphate (SSP) formulation for promoting bone and tissue growth in orthopedic applications which involve load bearing bones or ulcerous skin conditions which overcomes one or more of the shortcomings and limitations, set forth above.

According to an aspect of the invention, an SSP formulation is used to fix medical devices, such as, but not limited to prosthetic implants, by promoting direct soft tissue or bone adherence or in-growth to the prosthetic surface without intermediate tissue, such as granulation tissue, being formed.

According to a feature of the invention, the SSP formulation is used to achieve regeneration of tissue of the type to which it is contact. That is to say, for example, that when the SSP is in contact with muscle, muscle regenerates; when the SSP is in contact with skin, skin regenerates; when the SSP is in contact with vascular structures, vessels regenerate.

According to another aspect of the invention, because of the regenerative potentiation and anti-microbial characteristics the SSP formulation is used to treat diabetic and decubitus ulcers, traumatic injuries, such as motor vehicle accident injuries, and the like.

According to yet another aspect of the invention, the SSP formulation may be incorporated into various bioabsorbable fixation devices, such as, but not limited to plates, screws, anchors, staples, interference screws, rods, and the like, of various compositions, such as, but not limited to polylactic acid, polyglycolic acid, polyhydroxyalkonates, poliglecaprone, collagen preparations, and the like, to allow such devices to remodel into the host tissue type without the formation of granulation tissue and loosening.

According to another feature of the invention, application of the SSP formulation to bone soft tissue interfaces promotes the formation of Sharpey's fibers providing anatomic soft tissue to bone, load transferring bonds.

According to another feature of the invention, application of the SSP formulation to fractures with a high incidence of non or delayed union prevents non or delayed union. Again, the SSP formulation may be used in conjunction with other compatible vehicles or carriers for ease of application or injection, such as, but not limited to various biocompatible fluids or gels of various composition.

According to another feature of the invention, application of the SSP formulation to debrided bone cysts and benign bone tumors promotes the healing of the created defects with normal bone.

According to yet another aspect of the invention, the application of the SSP formulation as a paste composed of aqueous thrombin and precipitated collagen to a bleeding bone controls the bleeding and promotes normal bone healing.

Additional advantages of the invention include a commercially available, FDA approved supply of the SSP formulation which is easily handled and used in a sterile environment, such as an operating room requiring only application to involved areas, with or without the use of special apparatus. In addition, when used externally, the SSP formulation may be used by the patient to treat wounds or ulcers based on a doctor's instructions.

### DETAILED DESCRIPTION OF THE INVENTION

A commercially available silicon substituted phosphate (SSP) is marketed under the name Bioglas®, and in the studies described herein, the SSP designated 45S5 was used. The 45S5 name signifies an SSP with 45% SiO₂ and a 5 to 1 ratio of CaO to P₂O₅. Lower ratios of CaO to P₂O₅ have not promoted bonding with bone. More specifically the formulation by weight percent (wt %) of 45S5 is shown in (1).

45S5 45 SiO₂, 6 P₂O₅, 24.5 CaO, and 24.5 Na₂O (1)

A first study was performed which included 157 consecutive porous coated total hip and knee prostheses implanted using the SSP formulation designated 45S5 and without using methyl-methacrylate bone cement.

According to the method of the present invention, the bioactive SSP was sprinkled on all of the porous coated surfaces of the prosthetic device which had been wetted by normal saline/antibiotic solution. Additional SSP particles were mixed with autologous cancellous bone, and packed in all soft trabecular bone spaces, and in the sites adjacent to where the porous surfaces would be in contact. For larger defects additional autologous or bank cancellous bone was mixed with additional material and packed in the defects.

Retrospectively, 23 patients at high risk for loosening were sequentially selected for review without exclusion beginning with implants in place the longest. All high risk implants reviewed were implanted for a minimum of 7 years. None of the high risk patients or the remaining sequentially reviewed patients have had X-ray or clinical evidence of loosening. None of the patients experienced infections during the healing process. Bone pain was graded at 1 in 5, or less, in all patients, and in no instance did pain prevent activity or interfere with sleep. At grafted sites, X-rays confirmed new bone formation without resorbtion. At stem or “peg” ends the crescent shaped bone formation indicative of prosthetic micro-motion was usually absent, or if present, minimal. No radiolucency was noted adjacent to the surfaces coated with the material. In addition all soft tissue to bone attachment using the SSP modifying the growth and differentiation factor sequence were successful.

Because the SSP effectively seals all bone and soft tissue interfaces between bone and metal prosthetic components, partially by promoting ionic surface modification on metal, and enhancing soft tissue adhesion, no particulate loosening has been observed.

The anti-microbial character of the material discussed above, makes it ideally suited for orthopaedic implant use. It has been observed that silicon inhibits the glycoprotein “slime” formation of staph epidermis. Research studying SSP activity suggests that the silicon content by-passes the resorbtive or breakdown phase of inflammation thus promoting early new bone formation and imparts an anti-microbial activity. The tumor necrosis factor (TNF-α) and IL-1 are down-regulated and the vascular endothelial growth factor (VEGF) is up-regulated in the cellular growth and differentiation factor cascade.

Bone stimulation and early bone deposition also make the material well suited for other high risk patients with bone metabolic compromise, such as osteoporosis.

In another study a synthetic scaffold including the SSP designated 45S5 as shown in (1) seeded with autologous mesenchymal stem cells from adjacent or involved bone were implanted to replace a damaged anterior cruciate ligament (ACL), without failure in any cases of 23 cases. Various implant procedures for ACLs, were utilized, but all procedures had a tibial tunnel as the site of the mesenchymal stem cell development. The femoral attachment was accomplished through either a femoral tunnel or an “over-the-top” configuration, with soft tissue fixation (one tunnel). The former terminated in either soft tissue fixation; over a bone bridge; used an absorbable or non-absorbable interference screw, or a combination thereof. Arthroscopic or arthroscopically assisted, minimally open procedures were used for isolated ACL replacements. In some multi-trauma instances, the intra-articular ligament[s] (the ACL and/or PCL), and/or extra-articular ligament[s] (the MCL and/or the LCL) were replaced, also without failure in volunteers. The post-operative rehabilitation was minimally limiting due to the strength of the implant, but must be guarded to allow the in-growth tissue to mature in response to its environment.

In another study of approximately 15 patients presenting with fractures with a high incidence of non or delayed union. Application of the SSP formulation at the fracture was successful in healing the fracture without non or delayed union characteristics.

In another study of approximately 30 patients having debrided bone cysts and/or benign bone tumors were treated with a mixture of autologous cancellous bone and the SSP formulation designated in (1). Healing of the bone defects to normal bone was successful in all of the patients.

In another study involving more than 250 volunteers, the SSP formulation was used externally to promote tissue growth in ulcers or wounds that were difficult to heal because of inadequate blood flow to the area. The SSP formulation designated 45SP as seen in (1) was applied daily after cleaning the ulcerous area. The wound was covered loosely and kept dry. In all cases the participants reported that the ulcer or wound healing was improved when compared to treatment methods used for previous ulcers.

Because of the effectiveness of SSPs in anchoring prosthetic implants, the prevention of granulation particulates, the anti-microbial activity, the reconstituting soft tissue to bone interfaces, and its negligible production cost, SSP formulations, such as 45S5 may be used in most prosthetic implant procedures and soft tissue to bone repairs, particularly in high risk patients.

It is important to note that in most of the studies, the SSP formulation is applied internally typically by a doctor in an operating room. Once the incision is closed, the SSP formulation is remains inside the patient. When treating wounds or ulcerous tissue, the SSP formulation is applied externally, often by the patient. This use of the SSP is particularly advantageous because it can be offered “over the counter” directly to patients. It allows the patient to apply the SSP to treat the external ulcer according to a doctor's orders, most likely without hospitalization and even possibly without visiting a doctor.

According to a feature of the invention, the SSP may sprinkled or otherwise directly applied to involved areas or other components of treating modalities, such as but not limited to wound sites, prosthetics, tissue interfaces, debrided benign bone cysts and tumors, and the like.

According to another feature of the invention, the SSP formulation may be used in combination with a vehicle or carrier to facilitate the presentment of the invention to various sites, in some instances to less accessible areas, for example, by injection.

According to yet another feature of the inventions, the SSP formulation may be combined in various proportions with other materials to facilitate remodeling of various bioabsorbable devices.

The SSP used in the method of the invention is a sterile, dry composite which may be applied directly to open or accessible sites or may be otherwise deposited in a sol-gel derived material.

Because of the effectiveness of 45S5 SSP formulation in anchoring prosthetic implants, the prevention of granulation tissue, anti-microbial activity, reconstituting soft tissue to bone interfaces, and its availability, SSP formulations may be used in most prosthetic implant procedures and soft tissue to bone repairs. In addition the 45S5 SSP formulation may be used to promote soft tissue growth in ulcerous wounds that are otherwise difficult to heal because of diminished blood flow.

Advantages of the method of use of SSP formulations to improve healing of bone and soft tissue include use of an FDA approved substance that is commercially available. Use of the SSP collectively provides a sterile, surgically implantable material for the regeneration of living tissues of various composition, to allow tissue fixation to dissimilar tissue and other materials, to treat various medical conditions, and to promote clotting. This material chemically modulates various cellular growth and differentiation factors such as, but not exclusive to tumor necrosis factor, to promote tissue regeneration of the anatomic type that it is placed in contact with, while exhibiting antimicrobial activity.

In light of all the foregoing, it should thus be apparent to those skilled in the art that there has been shown and described a method of use of SSP to improve healing of bone and soft tissue. However, it should also be apparent that, within the principles and scope of the invention, many changes are possible and contemplated, including in the details, materials, and arrangements of parts which have been described to explain the nature of the invention. Thus, while the foregoing description and discussion addresses certain preferred embodiments or elements of the invention, it should further be understood that concepts of the invention, as based upon the foregoing description and discussion, may be readily incorporated into or employed in other embodiments and constructions without departing from the scope of the invention. Accordingly, the following claims are intended to protect the invention broadly as well as in the specific form shown, and all changes, modifications, variations, and other uses and applications which do not depart from the spirit and scope of the invention are deemed to be covered by the invention, which is limited only by the claims which follow.

What is claimed is:

1. A method to promote healing of an ulcer having a depressed surface due to tissue erosion bound by a perimeter using a silicon substituted phosphate formulation, the method comprising:
removing necrotic tissue from the depressed surface exposing reddened tissue;
cleansing the surface with a sterile solution;
sprinkling a layer having a thickness of approximately 200 to 500 micrometers of silicon substituted phosphate granules on the reddened tissue such that substantially all of the redness is masked by the layer; and
covering the surface with a sterile bandage;
wherein the silicon substituted phosphate formulation comprises a composition by molecular weight of approximately 40 to 50 percent SiO₂, approximately 2 to 8 percent P₂O₅, approximately 20 to 30 percent CaO, and the remaining molecular weight approximately 15 to 20 percent Na₂O, such that the ratio of CaO to P₂O₅ is greater than or equal to 5 to 1.

2. The method of claim 1, wherein the silicon substituted phosphate formulation promotes healing of the ulcer by encouraging regeneration of soft tissue.

3. The method of claim 1, wherein the silicon substituted phosphate formulation modifies cellular growth and differentiation factors and promotes secretion of extracellular matrix and biochemical factors to regulate genes of a host tissue to promote tissue regeneration of appropriate anatomic tissue when applied.

4. The method of claim 1, wherein the silicon substituted phosphate formulation exhibits anti-microbial activity while promoting soft tissue regeneration.

5. The method of claim 1, wherein the silicon substituted phosphate formulation is a sol-gel derived material and is deposited in a thin layer on the reddened tissue such that substantially all of the redness is masked by the thin layer.

6. The method of claim 1, wherein the silicon substituted phosphate formulation is directly applied to the ulcer surface or admixed with a vehicle or carrier for application to the ulcer surface.
